SEQUENCE LISTING (2 sequences)

SEQ ID NO: 1 - DNA, 49 bp, synthetic construct
  misc_feature 1..49: note = The sequence is synthesized.
  source 1..49: mol_type = other DNA
aggtcagatgaggagggggacttaggactgggtttatgacctatgcgtg

SEQ ID NO: 2 - DNA, 58 bp, synthetic construct
  misc_feature 1..58: note = modified base=NH2-a
  source 1..58: mol_type = other DNA
aaaaaaaaaaggtcagatgaggagggggacttaggactgggtttatgacctatgcgtg

(19) United States
(12) Patent Application Publication
(10) Pub. No.: US 2023/0201424 A1
(43) Pub. Date: Jun. 29, 2023

(54) TEMPERATURE-SENSITIVE GEL MATERIAL COMBINATION, PREPARATION METHOD THEREFOR AND USE THEREOF

(71) Applicant: THE FIRSTAFFILIATED HOSPITAL OF JINAN UNIVERSITY(GUANGZHOU OVERSEAS CHINESE HOSPITAL), Guangzhou (CN)

(72) Inventors: Xiangning LIU, Guangzhou (CN); Mingxiang CAI, Guangzhou (CN); Zinan XU, Guangzhou (CN); Yan LIANG, Guangzhou (CN)

(73) Assignee: THE FIRSTAFFILIATED HOSPITAL OF JINAN UNIVERSITY(GUANGZHOU OVERSEAS CHINESE HOSPITAL), Guangzhou (CN)

(21) Appl. No.: 18/062,584

(22) Filed: Dec. 7, 2022

(30) Foreign Application Priority Data
Dec. 17, 2021 (CN) 202111555471.5

(51) Int. Cl. A61L 27/48; A61L 27/52; A61L 27/54

(52) U.S. Cl. CPC A61L 27/48; A61L 27/52; A61L 27/54; A61L 2430/12; A61L 2300/414; A61L 2430/34

(57) ABSTRACT

A temperature-sensitive hydrogel material combination, a preparation method thereof, and use thereof are provided. The temperature-sensitive gel material combination provided by the present invention includes an SDF-1-loaded chitosan-hyaluronic acid solution and an Apt19S-loaded chitosan-hyaluronic acid solution. The present invention aims to develop a temperature-sensitive injectable double-layer hydrogel loaded with biological factors capable of promoting cell migration, which is used for promoting the regeneration of periodontal tissues.

### CROSS REFERENCE TO THE RELATED APPLICATIONS

This application is based upon and claims priority to Chinese Patent Application No. 202111555471.5, filed on Dec. 17, 2021, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBRZBC064_Sequence_Listing.xml, created on Jan. 10, 2023, and is 3,759 bytes in size.

### TECHNICAL FIELD

The present invention relates to the technical field of regenerative medicine, in particular to a temperature-sensitive gel material combination, a preparation method therefor and use thereof.

### BACKGROUND

Chronic periodontitis is a clinically common chronic inflammatory disease with progressive destruction of periodontal supporting tissues as the main pathological change, which can lead to tooth loosening and even loss in severe cases. Existing treatments such as supragingival scaling and subgingival scaling can only control disease progression, rather than reconstruct destroyed periodontal tissue. Therefore, many scholars expect to realize regeneration of periodontal tissues by means of tissue engineering, and provide a new idea for treating chronic periodontitis.

Seed cells, as one of the three elements of tissue engineering, are basic conditions for tissue regeneration. However, in vitro culture of allogeneic cells followed by transplantation often results in an immune response in the body, which affects the final regenerative and reparative effect. In addition, the in vitro culture followed by transplantation has complicated procedures and high costs. Therefore, endogenous regeneration has received increasing attention to promote periodontal tissue repair. Endogenous regeneration, namely, the recruitment of stem cells to periodontal defect sites through chemokines and the promotion of proliferation and differentiation of the stem cells, is expected to provide a new strategy for the physiological and functional reconstruction of periodontal tissues. The periodontal tissues comprise at least three components: cementum, periodontal ligament and alveolar bone. However, the current periodontal tissue regeneration scaffolds are mainly single-layer scaffolds, which lack adaptation to the complex physiological structure of the periodontal tissues.

As another important factor of tissue engineering, the scaffold materials need to be approximately matched with the morphology of the tissue defect region, so as to provide a suitable environment for cell growth and tissue generation. With the continuous progress and development of the technology, the 3D printing technology is widely applied to the development of scaffold materials, and the printed scaffold shows excellent performance. However, in order to ensure the adaptation to defects in different situations, the scaffold needs to be precisely designed according to actual situations, which adds additional costs; furthermore, the complex scaffold fabrication process and the frequent need for invasive scaffold implantation make 3D printing technically demanding.

### SUMMARY

An objective of the present invention is to provide a double-layer scaffold capable of directionally recruiting different cells in a layered manner, so as to realize the simultaneous regeneration of periodontal ligament and alveolar bone.

In order to achieve the above objective, the present invention provides the following technical schemes.

The present invention provides a temperature-sensitive gel material combination, which comprises an SDF-1-loaded chitosan-hyaluronic acid solution and an Apt19S-loaded chitosan-hyaluronic acid solution.

Preferably, the SDF-1-loaded chitosan-hyaluronic acid solution and the Apt19S-loaded chitosan-hyaluronic acid solution are individually packaged.

Preferably, the temperature-sensitive gel material combination is transformed from a solution state to a gel state at a temperature of 36-38° C.

The present invention also provides a preparation method for the temperature-sensitive gel material combination, which comprises the following steps:

(1) mixing chitosan with an acetic acid solution to obtain a chitosan solution;
(2) mixing β-glycerophosphate disodium with hyaluronic acid and dissolving the mixture in water to obtain a hyaluronic acid solution;
(3) mixing the chitosan solution with the hyaluronic acid solution to obtain a chitosan-hyaluronic acid solution;
(4) mixing SDF-1 with 40%-60% by volume of the chitosan-hyaluronic acid solution to obtain an SDF-1-loaded chitosan-hyaluronic acid solution; and
(5) mixing 40%-60% by volume of the chitosan-hyaluronic acid solution with 1-ethyl-(3-dimethylaminopropyl)carbodiimide and N-hydroxysuccinimide, and adding an Apt19S aptamer to obtain an Apt19S-loaded chitosan-hyaluronic acid solution.

Preferably, the acetic acid solution has a concentration of 0.08-0.12 M, and the chitosan and the acetic acid solution are in a mass-to-volume ratio of 28-32 mg:0.8-1.2 mL.

Preferably, the β-glycerophosphate disodium, the hyaluronic acid and the water are in a mass-to-volume ratio of 55-65 mg:0.8-1.2 mg:0.05-0.15 mL.

Preferably, the SDF-1 in the SDF-1-loaded chitosan-hyaluronic acid solution has a concentration of 180-230 ng/mL.

Preferably, the 1-ethyl-(3-dimethylaminopropyl)carbodiimide, the N-hydroxysuccinimide, and the chitosan-hyaluronic acid solution in the step (4) are in a mass-to-volume of 15-25 mg:8-12 mg:0.8-1.2 mL.

Preferably, the Apt19S aptamer in the Apt19S-loaded chitosan-hyaluronic acid solution has a concentration of 1.5-2.5 nmol/mL.

The present invention also provides use of the temperature-sensitive gel material combination in the preparation of a periodontal tissue regeneration scaffold.

Preferably, the periodontal tissue regeneration scaffold is of a double-layer gel structure;

in the double-layer gel structure, an SDF-1 gel formed by the SDF-1-loaded chitosan-hyaluronic acid solution is tightly abutted to an Apt19S gel formed by the Apt19S-loaded chitosan-hyaluronic acid solution to form an abutted surface;

a surface of the SDF-1 gel opposite to the abutted surface is close to the periodontal ligament, and a surface of the Apt19S gel opposite to the abutted surface is close to the alveolar bone.

The present invention provides a temperature-sensitive gel material combination. In the present invention, chitosan (CS), hyaluronic acid (HA) and β-glycerophosphate disodium (β-GP) are used as starting materials to obtain a chitosan-hyaluronic acid solution (CS/HA/GP solution), and then SDF-1 is added dropwise or after the carboxyl group on the hyaluronic acid is activated, an aptamer Apt19S is added, so as to obtain the temperature-sensitive gel material combination. The chitosan, hyaluronic acid, β-GP and other starting materials used for the gel of the present invention are inexpensive and readily available, the process of loading the drug is simple, and the production period is short. The temperature-sensitive gel material provided by the present invention can maintain at a liquid state and keep gelation ability for a relatively long time when stored at 4° C. It still has good fluidity and can be injected as an injection into periodontal tissues (between periodontal ligament and alveolar bone) at 25° C., and is transformed into a gel state at the body temperature (37° C.), which can be well matched with complicated periodontal tissues.

When the temperature-sensitive gel material combination of the present invention is used, the SDF-1-loaded chitosan-hyaluronic acid solution and the Apt19S-loaded chitosan-hyaluronic acid solution are injected into the oral cavity in a layered manner, so that the SDF-1 gel is close to the periodontal ligament, the Apt19S gel is close to the alveolar bone, and thus the aim of constructing a double-layer gel scaffold in the oral cavity is achieved.

The temperature-sensitive gel material combination provided by the present invention forms a scaffold matched with the morphology of a defect region in a periodontal defect region in vivo and capable of forming sufficient support in a body temperature environment in a low-cost and simple manner, and can continuously release growth factors required for attracting cell migration, thereby providing support for final cell immigration and tissue regeneration.

The stromal cell-derived factor-1 (SDF-1) and the aptamer Apt19S both have the ability to recruit stem cells, and Apt19S can specifically bind to mesenchymal stem cells, which provides an important way for the directed recruitment of stem cells to the lesion area. SDF-1 is used at a position close to the periodontal ligament in the periodontal defect region to recruit periodontal ligament stem cells, so as to promote the formation of the periodontal ligament; and Apt19S is used on the side close to the alveolar bone to recruit bone marrow mesenchymal stem cells, so as to induce the regeneration of the alveolar bone. Therefore, the present invention provides a double-layer temperature-sensitive gel capable of slowly releasing SDF-1/Apt19S to recruit periodontal ligament stem cells and bone marrow mesenchymal stem cells, which is used for promoting the regeneration of periodontal tissues.

The present invention aims to develop a temperature-sensitive injectable double-layer hydrogel loaded with biological factors capable of promoting cell migration, which is used for promoting the regeneration of periodontal tissues. It has the following advantages:

1. With the characteristics of temperature sensitivity and injectability, the temperature-sensitive gel material can be conveniently and efficiently injected into and matched with the periodontal defect region.

2. With the advantage of formation into two layers, SDF-1 is used at a position close to the periodontal ligament in the periodontal defect region to recruit periodontal ligament stem cells (PDLSCs), so as to promote the formation of the periodontal ligament; Apt19S is used on the side close to the alveolar bone to recruit bone marrow mesenchymal stem cells (BMSCs), so as to promote the regeneration of the alveolar bone, ultimately achieving the co-generation of periodontal tissues with different components.

### BRIEF DESCRIPTION OF THE DRAWINGS

FIG. 1 shows the gel state of the temperature-sensitive gel material prepared in Example 1 at 37° C.;

FIG. 2 shows the liquid state of the temperature-sensitive gel material prepared in Example 1 at 25° C.;

FIG. 3 shows a scanning electron micrograph of the temperature-sensitive gel material prepared in Example 1 as a scaffold material;

FIGS. 4A-4C show the results of Transwell cell migration assay on rBMSCs;

FIGS. 5A-5C show the results of Transwell cell migration assay on rPDLSCs;

FIG. 6 shows a schematic view of the temperature sensitive gel material of the present invention as a periodontal tissue regeneration scaffold.

### DETAILED DESCRIPTION OF THE EMBODIMENTS

The present invention provides a temperature-sensitive gel material combination, which comprises an SDF-1-loaded chitosan-hyaluronic acid solution and an Apt19S-loaded chitosan-hyaluronic acid solution.

In the present invention, the SDF-1-loaded chitosan-hyaluronic acid solution and the Apt19S-loaded chitosan-hyaluronic acid solution are preferably individually packaged.

In the present invention, the temperature-sensitive gel material combination is preferably transformed from a solution state to a gel state at a temperature of 36-38° C., and further preferably 37° C.

In the present invention, the temperature-sensitive gel material combination is preferably an injection.

The present invention also provides a preparation method for the temperature-sensitive gel material combination, which comprises the following steps:

(1) mixing chitosan with an acetic acid solution to obtain a chitosan solution;
(2) mixing β-glycerophosphate disodium with hyaluronic acid and dissolving the mixture in water to obtain a hyaluronic acid solution;
(3) mixing the chitosan solution with the hyaluronic acid solution to obtain a chitosan-hyaluronic acid solution;
(4) mixing SDF-1 with 40%-60% by volume of the chitosan-hyaluronic acid solution to obtain an SDF-1-loaded chitosan-hyaluronic acid solution; and
(5) mixing 40%-60% by volume of the chitosan-hyaluronic acid solution with 1-ethyl-(3-dimethylaminopropyl)carbodiimide and N-hydroxysuccinimide, and adding an Apt19S aptamer to obtain an Apt19S-loaded chitosan-hyaluronic acid solution.

In the present invention, chitosan and an acetic acid solution is mixed to obtain a chitosan solution.

In the present invention, the acetic acid solution preferably has a concentration of 0.08-0.12 M, and further preferably 0.1 M.

In the present invention, the chitosan (CS) and the acetic acid solution are preferably in a mass-to-volume ratio of 28-32 mg:0.8-1.2 mL, and further preferably 30 mg:1 mL.

In the present invention, the chitosan is preferably mixed with the acetic acid solution by stirring and mixing with a magnetic stirrer.

In the present invention, the magnetic stirrer preferably has a rotation speed of 800-1200 rpm, and further preferably 1000 rpm.

In the present invention, the stirring time for the magnetic stirrer is preferably 1.5-2.5 h, and further preferably 2 h.

In the present invention, β-glycerophosphate disodium (β-GP) and hyaluronic acid (HA) are dissolved in water to obtain a hyaluronic acid solution.

In the present invention, the β-glycerophosphate disodium, the hyaluronic acid and the water are preferably in a mass-to-volume ratio of 55-65 mg:0.8-1.2 mg:0.05-0.15 mL, further preferably 58-62 mg:0.9-1.1 mg:0.08-0.12 mL, and still further preferably 60 mg:1 mg:0.1 mL.

In the present invention, the water is preferably deionized water.

The obtained chitosan solution is mixed with a hyaluronic acid solution to obtain the chitosan-hyaluronic acid solution.

In the present invention, the chitosan solution and the hyaluronic acid solution are preferably subjected to an ice-bath treatment before being mixed.

In the present invention, the ice-bath treatment is performed at a temperature of preferably −2-4° C., and further preferably 0° C.

In the present invention, the time for the ice-bath treatment is preferably 8-12 min, and further preferably 10 min.

In the present invention, the hyaluronic acid solution is preferably added dropwise to the chitosan solution while the chitosan solution is mixed with the hyaluronic acid solution.

In the present invention, the chitosan solution is mixed with the hyaluronic acid solution by stirring preferably using a magnetic stirrer.

In the present invention, the magnetic stirrer preferably has a rotation speed of 500-1500 rpm, further preferably 800-1200 rpm, and still further preferably 1000 rpm.

In the present invention, the time for the magnetic stirrer is preferably 8-12 min, and further preferably 10 min.

In the present invention, the obtained chitosan-hyaluronic acid solution is divided into two parts. SDF-1 is mixed with 40%-60% by volume of a chitosan-hyaluronic acid solution to obtain the SDF-1-loaded chitosan-hyaluronic acid solution.

In the present invention, the chitosan-hyaluronic acid solution is preferably divided into two equal parts.

In the present invention, the SDF-1 is preferably added dropwise to the chitosan-hyaluronic acid solution, and magnetically stirred during the dropwise addition.

In the present invention, the magnetic stirring is preferably performed at a rotation speed of 500-1500 rpm, further preferably 800-1200 rpm, and still further preferably 1000 rpm.

In the present invention, the time for the magnetic stirrer is preferably 8-12 min, and further preferably 10 min.

In the present invention, the SDF-1 in the SDF-1-loaded chitosan-hyaluronic acid solution preferably has a concentration of 180-230 ng/mL, further preferably 190-210 ng/mL, and still further preferably 200 ng/mL.

In the present invention, the other part of 40%-60% by volume of the chitosan-hyaluronic acid solution is mixed with 1-ethyl-(3-dimethylaminopropyl)carbodiimides and N-hydroxysuccinimide, followed by the addition of an Apt19S aptamer to obtain the Apt 19S-loaded chitosan-hyaluronic acid solution.

In the present invention, preferably, the chitosan-hyaluronic acid solution is diluted with an activation buffer and then mixed with 1-ethyl-(3-dimethylaminopropyl)carbodiimide and N-hydroxysuccinimide.

In the present invention, the dilution is preferably at a fold of ¼-⅕, and further preferably ⅕.

In the present invention, the activation buffer is preferably an IVIES buffer.

In the present invention, the MES buffer preferably has a concentration of 0.05-0.15 M, and further preferably 0.1 M.

In the present invention, the IVIES buffer preferably has a pH of 5.8-6.3, and further preferably 6.0.

In the present invention, the 1-ethyl-(3-dimethylaminopropyl)carbodiimide, the N-hydroxysuccinimide and the chitosan-hyaluronic acid solution are preferably in a mass-to-volume ratio of 15-25 mg:8-12 mg:0.8-1.2 mL, further preferably 18-22 mg:9-11 mg:0.8-1.1 mL, and still further preferably 20 mg:10 mg:1 mL.

In the present invention, after 1-ethyl-(3-dimethylaminopropyl)carbodiimide and N-hydroxysuccinimide are added to the diluted chitosan-hyaluronic acid solution, the mixture is preferably left to stand for 12-18 min, and further preferably left to stand for 15 min, followed by the addition of the Apt19S aptamer. In the present invention, the 1-ethyl-(3-dimethylaminopropyl)carbodiimide and the N-hydroxysuccinimide act to activate the carboxyl group on hyaluronic acid in a chitosan-hyaluronic acid solution.

In the present invention, the Apt19S aptamer has a nucleotide sequence of 5′-AGGTCAGATGAGGAGGGGGACTTAGGACTGGGTTTATGACCTATGCGTG-3′ (as set forth in SEQ ID NO: 1).

In the present invention, the Apt19S aptamer is further preferably an amino-modified Apt19S aptamer.

In the present invention, the amino-modified Apt19S aptamer has a nucleotide sequence of 5′-NH₂-(A)₉-AGGTCAGATGAGGAGGGGGACTTAGGACTGGGTTTATGACCTATGCGTG-3′ (as set forth in SEQ ID NO: 2).

In the present invention, the Apt19S aptamer is preferably added dropwise.

In the present invention, after the Apt19S aptamer is added, the mixture is preferably mixed by shaking for 10-15 h, and further preferably for 12 h.

In the present invention, the Apt19S aptamer in the Apt19S-loaded chitosan-hyaluronic acid solution preferably has a concentration of 1.5-2.5 nmol/mL, further preferably 1.8-2.2 nmol/mL, and still further preferably 2 nmol/mL.

The present invention also provides use of the temperature-sensitive gel material combination in the preparation of a periodontal tissue regeneration scaffold.

In the present invention, the periodontal tissue regeneration scaffold is preferably of a double-layer gel structure.

In the present invention, in the double-layer gel structure, preferably, an SDF-1 gel formed by the SDF-1-loaded chitosan-hyaluronic acid solution is tightly abutted to an Apt19S gel formed by the Apt19S-loaded chitosan-hyaluronic acid solution to form an abutted surface.

In the present invention, a surface of the SDF-1 gel opposite to the abutted surface is preferably close to the periodontal ligament, and a surface of the Apt19S gel opposite to the abutted surface is preferably close to the alveolar bone.

When the temperature-sensitive gel material combination of the present invention is used as a periodontal tissue regeneration scaffold, firstly, the SDF-1-loaded chitosan-hyaluronic acid solution is injected into a position close to a periodontal ligament, and then the SDF-1-loaded chitosan-hyaluronic acid solution can form an SDF-1 gel at the human body temperature (37° C.); when the initial phase transition of the SDF-1-loaded chitosan-hyaluronic acid solution occurs, the Apt19S-loaded chitosan-hyaluronic acid solution is injected into a position under the SDF-1 gel and close to the alveolar bone, and the Apt19S-loaded chitosan-hyaluronic acid solution can also form an Apt19S gel at the human body temperature (37° C.); in the process of forming stable gels by the SDF-1-loaded chitosan-hyaluronic acid solution and the Apt 19S-loaded chitosan-hyaluronic acid solution, respectively, a tightly connected abutted surface is formed at the boundary position of the SDF-1-loaded chitosan-hyaluronic acid solution and the Apt19S-loaded chitosan-hyaluronic acid solution, and the double-layer structure is the periodontal tissue double-layer gel regeneration scaffold.

The technical schemes provided by the present invention will be described in detail below with reference to the examples, which, however, should not be construed as limiting the scope of the present invention.

### Example 1

48 mg of chitosan (CS) was dissolved in 1.6 mL of 0.1 M acetic acid solution, and the solution was stirred continuously for 2 h on a magnetic stirrer (1000 rpm) and sterilized by autoclaving at 121° C. for 10 min to obtain a chitosan solution. 240 mg of β-GP and 4 mg of hyaluronic acid were dissolved in 0.4 mL of deionized water, and the solution was filtered and sterilized with a 0.22 μm filter to obtain a hyaluronic acid solution. The chitosan solution and the hyaluronic acid solution were cooled in an ice bath (0° C.) for 15 min, then the hyaluronic acid was added dropwise to the chitosan solution, and the mixture was stirred for 10 min by a magnetic stirrer (1000 rpm) to obtain a chitosan-hyaluronic acid solution. The chitosan-hyaluronic acid solution was divided equally into two parts, each of 1 mL.

One part of 1 mL of the chitosan-hyaluronic acid solution was taken, 1 μL of a solution of SDF-1 in PBS at the concentration of 100 μg/mL was added dropwise thereto, and the mixture was magnetically stirred for 10 min (1000 rpm) to obtain an SDF-1-loaded chitosan-hyaluronic acid solution at the concentration of 200 ng/mL.

The other part of 1 mL of the chitosan-hyaluronic acid solution was taken and diluted with 200 μL of an activation buffer (0.1 M IVIES, pH=6), 20 mg of EDC and 10 mg of NHS were added at room temperature (20° C.), mixed, and left to stand at room temperature (20° C.) for 15 min to activate the carboxyl group on the hyaluronic acid. Then, 300 μL of a solution of 3 nmol amino-modified Apt19S in PBS was added dropwise, and the mixture was shaken on a shaker for 12 h for fully crosslinking to obtain an Apt19S-loaded chitosan-hyaluronic acid solution. The amino-modified Apt19S in the Apt19S-loaded chitosan-hyaluronic acid solution has a concentration of 2 nmol/mL.

In this example, the amino-modified Apt19S has a sequence of 5′-NH₂-(A)₉-AGGTCAGATGAGGAGGGGGACTTAGGACTGGGTTTATGACCTATGCGTG-3′ (as set forth in SEQ ID NO: 2).

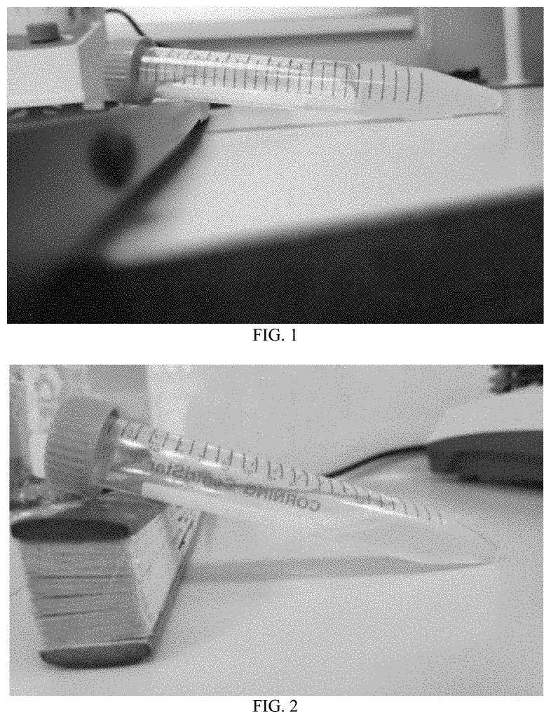

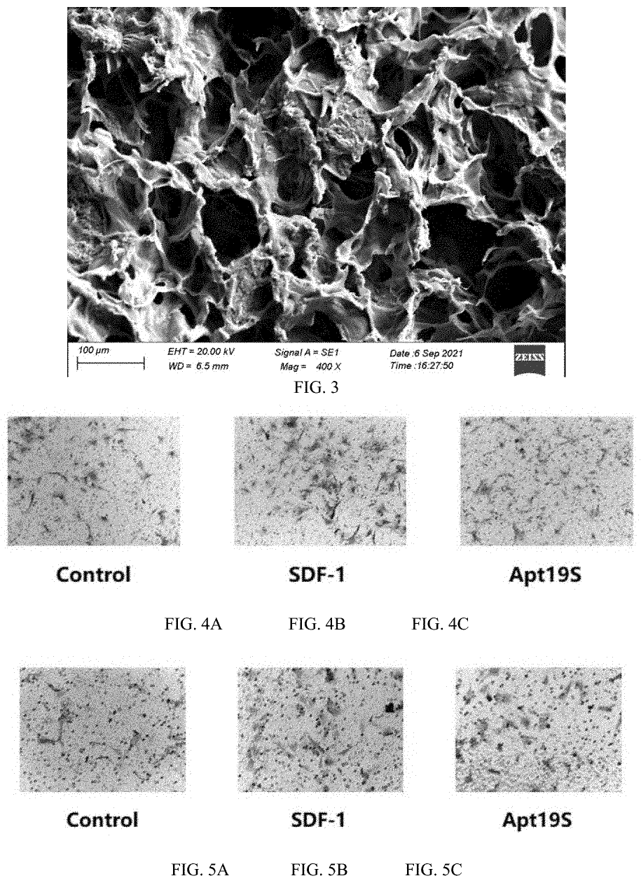

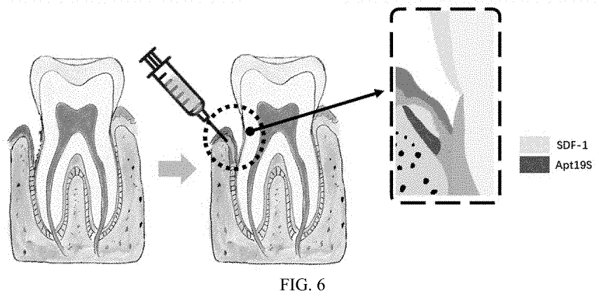

Two 5 mL syringes were connected to a three-way tube and were connected to each other, and 1 mL of the SDF-1-loaded chitosan-hyaluronic acid solution and 1 mL of the Apt19S-loaded chitosan-hyaluronic acid solution were added to the two syringes, respectively. The SDF-1-loaded chitosan-hyaluronic acid solution was injected into a 5 mL centrifuge tube. Then, the centrifuge tube was left to stand in a water bath at 37° C. for 2 min to cause the initial phase transition in the solution, then the Apt19S-loaded chitosan-hyaluronic acid solution in the other syringe was injected onto the ungelated solution in the centrifuge tube, which was then left to stand in a water bath at 37° C. for 10 min to obtain the double-layer hydrogel (as shown in FIG. 1). The temperature-sensitive gel material of the present invention showed a good liquid state at 25° C. (as shown in FIG. 2), and the morphology and pore size of the gel were observed under a scanning electron microscope (as shown in FIG. 3). As can be seen from FIG. 3, the double-layer gel scaffold is porous and loose, has a pore size of 80-100 and is suitable for cell climbing and cell growth.

### Example 2

100 μL of a culture medium containing the 3rd generation of rBMSCs (containing 1×10³ cells) was inoculated into the upper chamber of a Transwell chamber, and 600 μL of α-MEM medium containing 200 ng/mL SDF-1 and 600 μL of α-MEM medium containing amino-modified Apt19S at a concentration of 2 nmol/mL were added to the lower chambers of the Transwell chamber, respectively. After culturing in an incubator at 37° C. with 5% CO₂ for 24 h, cells in the upper chamber which did not migrate were wiped off with a cotton swab, and cells which migrated to the lower chamber were immobilized with 4% formaldehyde for 15 min, washed twice with PBS, dried, stained with 0.5% crystal violet for 30 min and washed 3 times with PBS. Then, 5 fields of view were randomly selected from each chamber under the microscope, and the number of BMSC cells migrated into the lower chamber was counted. The results show that: the addition of SDF-1 and the amino-modified Apt19S aptamer can significantly promote the migration of BMSC cells as compared to the blank group (as shown in FIGS. 4A-4C).

### Example 3

100 μL of a culture medium containing the 3rd generation of rPDLSCs (containing 1×10³ cells) was inoculated into the upper chamber of a Transwell chamber, and 600 μL of α-MEM medium containing 200 ng/mL SDF-1 and 600 μL of α-MEM medium containing Apt19S at a concentration of 2 nmol/mL were added to the lower chambers of the Transwell chamber, respectively. After culturing in an incubator at 37° C. with 5% CO₂ for 24 h, cells in the upper chamber which did not migrate were wiped off with a cotton swab, and cells which migrated to the lower chamber were immobilized with 4% formaldehyde for 15 min, washed twice with PBS, dried, stained with 0.5% crystal violet for 30 min and washed 3 times with PBS. Then, 5 fields of view were randomly selected from each chamber under the microscope, and the number of PDLSCs migrated into the lower chamber was counted. The results show that: the addition of SDF-1 and Apt19S can significantly promote the migration of PDLSCs as compared to the blank group (as shown in FIGS. 5A-5C).

The above descriptions are only preferred embodiments of the present invention. It should be noted that those of ordinary skill in the art can also make several improvements and modifications without departing from the principle of the present invention, and such improvements and modifications shall fall within the protection scope of the present invention.

SEQUENCE LISTING

| | |
|---|---|
| <110> | First Affiliated Hospital of Jinan University |
| <120> | TEMPERATURE-SENSITIVE GEL MATERIAL COMBINATION, |
| | PREPARATION METHOD THEREFOR AND USE THEREOF |
| <160> | 2 |
| <170> | SIPOSequenceListing 1.0 |
| <210> | 1 |
| <211> | 49 |
| <212> | DNA |
| <213> | Artificial Sequence |
| <400> | 1 |

| | |
|---|---|
| aggtcagatg aggaggggga cttaggactg ggtttatgac ctatgcgtg | 49 |

| | |
|---|---|
| <210> | 2 |
| <211> | 58 |
| <212> | DNA |
| <213> | Artificial Sequence |
| <220> | |
| <221> | modified_base |
| <222> | (1)..(1) |
| <223> | modified base = NH2-a |
| <400> | 2 |

| | |
|---|---|
| aaaaaaaaaa ggtcagatga ggagggggac ttaggactgg gtttatgacc tatgcgtg | 58 |

What is claimed is:

1. A temperature-sensitive gel material combination, comprising an SDF-1-loaded chitosan-hyaluronic acid solution and an Apt19S-loaded chitosan-hyaluronic acid solution, wherein the temperature-sensitive gel material combination is prepared as follows:
(1) mixing chitosan with an acetic acid solution to obtain a chitosan solution;
(2) mixing β-glycerophosphate disodium with hyaluronic acid to obtain a mixture and dissolving the mixture in water to obtain a hyaluronic acid solution;
(3) mixing the chitosan solution with the hyaluronic acid solution to obtain a chitosan-hyaluronic acid solution;
(4) mixing SDF-1 with 40%-60% by volume of the chitosan-hyaluronic acid solution to obtain the SDF-1-loaded chitosan-hyaluronic acid solution; and
(5) mixing 40%-60% by volume of the chitosan-hyaluronic acid solution with 1-ethyl-(3-dimethylaminopropyl)carbodiimide and N-hydroxysuccinimide, and adding an Apt19S aptamer to obtain the Apt19S-loaded chitosan-hyaluronic acid solution.

2. The temperature-sensitive gel material combination according to claim 1, wherein the SDF-1-loaded chitosan-hyaluronic acid solution and the Apt19S-loaded chitosan-hyaluronic acid solution are individually packaged; and the temperature-sensitive gel material combination is transformed from a solution state to a gel state at a temperature of 36-38° C.

3. A preparation method for a temperature-sensitive gel material combination, comprising the following steps:
(1) mixing chitosan with an acetic acid solution to obtain a chitosan solution;
(2) mixing β-glycerophosphate disodium with hyaluronic acid to obtain a mixture and dissolving the mixture in water to obtain a hyaluronic acid solution;
(3) mixing the chitosan solution with the hyaluronic acid solution to obtain a chitosan-hyaluronic acid solution;
(4) mixing SDF-1 with 40%-60% by volume of the chitosan-hyaluronic acid solution to obtain an SDF-1-loaded chitosan-hyaluronic acid solution; and
(5) mixing 40%-60% by volume of the chitosan-hyaluronic acid solution with 1-ethyl-(3-dimethylaminopropyl)carbodiimide and N-hydroxysuccinimide, and adding an Apt19S aptamer to obtain an Apt19S-loaded chitosan-hyaluronic acid solution.

4. The preparation method according to claim 3, wherein the acetic acid solution has a concentration of 0.08-0.12 M, and the chitosan and the acetic acid solution are in a mass-to-volume ratio of 28-32 mg:0.8-1.2 mL.

5. The preparation method according to claim 4, wherein the β-glycerophosphate disodium, the hyaluronic acid, and the water are in a mass-to-volume ratio of 55-65 mg:0.8-1.2 mg:0.05-0.15 mL.

6. The preparation method according to claim 5, wherein the SDF-1 in the SDF-1-loaded chitosan-hyaluronic acid solution has a concentration of 180-230 ng/mL.

7. The preparation method according to claim 6, wherein the 1-ethyl-(3-dimethylaminopropyl)carbodiimide, the N-hydroxysuccinimide, and the chitosan-hyaluronic acid solution in the step (5) are in a mass-to-volume ratio of 15-25 mg:8-12 mg:0.8-1.2 mL.

8. The preparation method according to claim 3, wherein the Apt19S aptamer in the Apt19S-loaded chitosan-hyaluronic acid solution has a concentration of 1.5-2.5 nmol/mL.

9. A method of a use of the temperature-sensitive gel material combination according to claim 1 in a preparation of a periodontal tissue regeneration scaffold.

10. The method according to claim 9, wherein the periodontal tissue regeneration scaffold is of a double-layer gel structure;
in the double-layer gel structure, an SDF-1 gel formed by the SDF-1-loaded chitosan-hyaluronic acid solution is tightly abutted to an Apt19S gel formed by the Apt19S-loaded chitosan-hyaluronic acid solution to form an abutted surface;
a surface of the SDF-1 gel opposite to the abutted surface is close to a periodontal ligament, and a surface of the Apt19S gel opposite to the abutted surface is close to an alveolar bone.

11. The preparation method according to claim 4, wherein the Apt19S aptamer in the Apt19S-loaded chitosan-hyaluronic acid solution has a concentration of 1.5-2.5 nmol/mL.

12. The preparation method according to claim 5, wherein the Apt19S aptamer in the Apt19S-loaded chitosan-hyaluronic acid solution has a concentration of 1.5-2.5 nmol/mL.

13. The preparation method according to claim 6, wherein the Apt19S aptamer in the Apt19S-loaded chitosan-hyaluronic acid solution has a concentration of 1.5-2.5 nmol/mL.

14. The preparation method according to claim 7, wherein the Apt19S aptamer in the Apt19S-loaded chitosan-hyaluronic acid solution has a concentration of 1.5-2.5 nmol/mL.

15. The method according to claim 9, wherein the SDF-1-loaded chitosan-hyaluronic acid solution and the Apt19S-loaded chitosan-hyaluronic acid solution are individually packaged; and the temperature-sensitive gel material combination is transformed from a solution state to a gel state at a temperature of 36-38° C.
